(19) United States
(12) Patent Application Publication
(10) Pub. No.: US 2019/0351076 A1
(43) Pub. Date: Nov. 21, 2019

(54) TUMOR CELL XENOGRAFT MODEL IN ZEBRAFISH, AND METHODS OF CONSTRUCTING AND USING THE SAME

(71) Applicant: NANJING EMORY BIOTECHNOLOGY CO., LTD., Nanjing, Jiangsu (CN)

(72) Inventors: Mingfang HE, Nanjing (CN); Ruixue WANG, Nanjing (CN); Jianying LI, Nanjing (CN)

(73) Assignee: NANJING EMORY BIOTECHNOLOGY CO., LTD., Nanjing, Jiangsu (CN)

(21) Appl. No.: 16/475,793

(22) PCT Filed: Dec. 27, 2017
(86) PCT No.: PCT/CN2017/118991

§ 371 Date: Jul. 3, 2019

(30) Foreign Application Priority Data
Jan. 22, 2017 (CN) 201710047294.7
Nov. 8, 2017 (CN) 201711091169.2

(51) Int. Cl. A61K 49/00; A01K 67/027

(52) U.S. Cl. CPC A61K 49/0008; A01K 67/0271; A01K 2267/0331; A01K 2227/40; A01K 2207/12

(57) ABSTRACT

A tumor cell xenograft model in zebrafish, and methods of constructing and using the same. Primary cells dissociated from the tumor tissue of a patient are transplanted into zebrafish, so as to obtain a patient-derived tumor xenograft model. The tumor xenograft model retains the pathological features of human gastric cancer tissues in clinic, has higher clinical relevance, and can be used in the systematic research into the mechanisms underlying the proliferation, metastasis, spread and drug resistance of tumors, and to screen effective drugs for tumor treatments.

### BACKGROUND

### Technical Field

The present invention relates to the technical field of biomedicine, and in particular to a patient-derived gastric cancer xenograft model in zebrafish, and methods of constructing and using the same.

### Related Art

Tumor diseases have become a major public health problem in the world. The most common tumors include lung cancer, gastric cancer, and breast cancer. Among them, gastric cancer is one of the most common malignant tumors of the digestive system found in the world, and has high incidence in East Asia. According to the “World Cancer Report 2014” published by the World Health Organization (WHO), the new cases of gastric cancer and deaths in China accounted for more than 40% of the total number in the whole world in 2012. In 2016, the Cancer Statistics in China, 2015 published in an authoritative journal “CA Cancer J Clin” sponsored by the American Cancer Society (ACS) showed that there were 679,100 new cases of gastric cancer in China in 2015, among them, the new cases of development in male patients were 477,700, ranking the second place among the high incidence of cancers in male patients, second only to the lung cancer. The new cases of development in female patients were 201,400, ranking the third place among the high incidence of cancers in female patients, second only to the breast cancer and lung cancer. Gastric cancer has become the second leading cause of death in the Chinese population, and there are 498,000 cases of death, second only to that caused by lung cancer.

After radical resection, the 5-year survival rate of patients with early gastric cancer can reach 90%. However, due to the non-obvious early symptoms of gastric cancer and the lack of popularization of routine gastroscopy, about 80% of patients with gastric cancer in China have reached the advanced stage upon clinical consultation. The existing treatments for gastric cancer are limited, and the overall survival rate of surgery alone is only about 20%. Radiotherapy and chemotherapy are often used for preoperative or postoperative adjuvant therapy. The drug treatment of gastric cancer is still dominated by typical chemotherapeutic agents, such as 5-fluorouracil, paclitaxel and platinum-based agents. Targeted drugs are still under clinical trials in the treatment of gastric cancer. Because gastric cancer is a highly heterogeneous tumor, many existing clinical evidences have shown that chemotherapy can prolong the survival time of patients with gastric cancer, but no “gold standard” therapeutic regimen with recognized obvious advantages and individualized medications has been found. Many patients lose their original treatment window because they fail to receive the drug that best matches the individual. Therefore, there is an urgent need for personalized guidance for medication regimen of gastric cancer in clinic.

In addition, lung cancer is the leading cause of malignant tumor-related death today. Epidemiological data shows that the global cases of death from lung cancer in 2012 were about 1.6 million, accounting for 19.4% of all deaths caused by malignant tumors. As shown by newly released statistical data in China, there are about 730,000 new cases of lung cancer, and about 610,000 cases of deaths in China in 2015. The incidence and mortality rates dominate other malignant tumors. The new cases and mortality of lung cancer in male patients ranks first among all malignant tumors. The new cases and mortality of lung cancer in female patients are significantly lower than that of male patients, and the new cases of development are ranked fourth (lower than breast cancer, colorectal cancer and cervical cancer), and the mortality ranks second (following breast cancer). Although the treatment of lung cancer has developed rapidly in recent years, the overall prognosis has not improved significantly. The current 5-year overall survival rate is only 16%-18%.

There are many types of lung cancer, the most common of which is non-small cell lung cancer (NSCLC), and because of the lack of specific symptoms in early stage of lung cancer, the condition has progressed to the middle or late stage when most patients are diagnosed, so the difficulty of treatment is increased. Non-small cell lung cancer accounts for more than 80% of the total lung cancer. Among the first diagnosed cases, 25% to 30% are locally advanced, and 40% to 50% have metastases. At present, chemotherapy for NSCLC is still based on the two-drug chemotherapy regimen of platinum agents combined with third-generation chemotherapeutic drugs recommended by the American Society of Clinical Oncology (ASCO) and the National Comprehensive Cancer Network (NCCN), such as gemcitabine+platinum agent, docetaxel+platinum agent, vinorelbine+platinumagent, and pemetrexed+platinum agent. For other types of lung cancer, the currently commonly used chemotherapeutic agents in clinic are cisplatin, gemcitabine, doxorubicin, paclitaxel, vincristine and the like. In addition, the epidermal growth factor receptor (EGFR)-TK inhibitor (TKI) is a small molecule inhibitor of EGFR targets in lung cancer, such as gefitinib, erlotinib and icotinib. Both afatinib and dacomitinib have entered the clinical phase, becoming a new effective and irreversible promising drug for the treatment of lung cancer.

Although there are many drugs for treating lung cancer, the survival rate of patients has not been improved significantly, and chemotherapy is palliative, mainly aiming at prolonging the survival of patients and improving their quality of life. At present, the commonly used anti-tumor chemotherapeutic drugs are less than 70% effective in the treatment of patients, and because of the lack of genetic analysis in individualized treatment with chemotherapy drugs, 20% to 40% of patients may even receive the incorrect drug treatment. Patients with the same pathological type, duration, and even the same molecular phenotype may have a “vastly different” outcome after receiving the same regimen of treatment. Therefore, the early screening of high-risk groups, the detection of types of gene mutation at the molecular level, and the search for a treatment regimen with personalized medication from various clinical treatment options are development directions of lung cancer treatment in future. Many patients lose their original treatment window because they fail to receive the drug that best matches the individual. Therefore, there is an urgent need for personalized guidance for medication regimen of lung cancer in clinic.

The patient-derived tumor xenograft (PDX) model is an immunodeficient animal that fresh tumor tissue of a patient is transplanted and grown in the microenvironment provided by the animal. Compared with the human-derived tumor cell line xenograft model, the differentiation degree, morphological features, structural characteristics and molecular properties of the PDX model tumors are closer to the tumor characteristics of the patients themselves, thus providing an important in vivo model for the biological research and seeking of diagnostic markers of tumors and for drug screening. In addition, the PDX model can reflect the tumor characteristics of the patient that the sample is derived, including the specificity of the drug response. Therefore, the PDX model has a higher clinical relevance than the traditional tumor cell line xenograft model, and has more important implications for the preclinical evaluation, treatment and prognosis of the tumors, and irreplaceable value especially for the individualized diagnosis and treatment of tumors. At present, mice are the most commonly used tumor PDX model animals. However, because the duration from tumor transplantation, tumor formation to efficacy evaluation in mice is usually 3 months, and many patients have a survival period of less than 3 months, the existing PDX model cannot meet the significant demands for clinical real-time guidance for individualized medication.

### SUMMARY

It is an object of the present invention to provide a patient-derived tumor cell xenograft animal model and a construction method and use thereof in the screening of therapeutic drugs for tumors.

The present invention provides a patient-derived tumor cell xenograft model in zebrafish, the embryos of which have been transplanted with primary single cells isolated and cultured from tumor tissue derived from a patient. Such tumors include, but are not limited to, solid tumors and blood born tumors, especially solid tumors, particularly lung cancer and gastric cancer.

The transplantation to the zebrafish embryos of the present invention is carried out within 24-72 hours, preferably within 36-60 hours, more preferably at 48 hours, after fertilization of the zebrafish. The transplantation site is in the yolk sac of the zebrafish embryos.

The primary single cells of the tumor tissue derived from the patient mentioned in the present invention are stained with a staining reagent before being transplanted into the embryos of the zebrafish, and the staining reagent is also called a dyeing dye, and selected from a fluorescent dye, preferably the fluorescent dye CM-Dil, having a concentration of 1-5 μg/ml.

Another aspect of the present invention provides use of the patient-derived tumor cell xenograft model in zebrafish in studying the mechanism underlying proliferation, metastasis, spread or drug resistance of tumors such as gastric cancer and lung cancer, or in screening effective therapeutic drugs for tumors such as gastric cancer and lung cancer. In particular, the tumor cell xenograft model in zebrafish of the present invention is particularly suitable for the study of tumor cell proliferation, and particularly the activity of therapeutic drugs. For the study of therapeutic effect of drugs, it is especially suitable for the study of therapeutic effects of 5-FU (5-fluorouracil) on patients with gastric cancer or gefitinib, cisplatin or docetaxel on patients with lung cancer alone or in combination.

The use of the patient-derived tumor cell xenograft model in zebrafish of the present invention in screening effective therapeutic drugs for tumors includes the steps of: determining the highest drug concentration within the safe range of a anti-tumor drug candidate for the untransplantated embryos; soaking the patient-derived tumor cell xenografted zebrafish embryos with the drug candidate having a drug concentration in the safe range; taking the solvent for dissolving the drug candidate as a control and undergoing the same process; and performing qualitative analysis or/and quantitative analysis of the proliferation and spread of the patient-derived cells in the zebrafish embryos under a fluorescence microscope. For the quantitative analysis, the antitumor effect is calculated according to the following formula: the tumor inhibition rate of the drug=(the fluorescence intensity of the treatment group/the fluorescence intensity of the control group)*100%. When the inhibition rate is less than 100%, it indicates that the drug has inhibition on tumors, and the smaller the value is, the more significant the tumor inhibition effect will be.

The time for soaking the zebrafish embryos with the drug candidate is 2 to 5 days, and preferably 3 days. The observation time is preferably at days 1, 4, and 7, or the observation and calculation may be done only on day 7.

The use of the patient-derived tumor cell xenograft model in zebrafish of the present invention in screening effective therapeutic drugs for tumors such as lung cancer and gastric cancer includes specifically the steps of:

(1) treating untransplanted zebrafish embryos one to three days and preferably two days after fertilization, with various concentrations of a drug candidate, for three to five days, and preferably four days, to determine the highest drug concentration within the safe range for the embryos according to the embryo survival;

(2) treating zebrafish embryos that have been injected with patient-derived gastric cancer cells 1 to 3 days and preferably two days after fertilization, with the drug candidate that the highest drug concentration within the safe range for the embryos has been determined, for 2 to 5 days, and preferably 3 days, and the solvent for the drug candidate is used as a control; and

(3) observing the proliferation and spread of red patient-derived cells in the zebrafish embryos after treatment. The red cells are photographed under a fluorescence microscope, the red fluorescence intensity is quantified using Image Pro Plus software, and the antitumor effect of the drug is calculated using a formula.

A third aspect of the present invention provides a method for constructing a patient-derived tumor (such as lung cancer, and gastric cancer) cell xenograft model in zebrafish, which includes the steps of:

(1) dissociating a patient-derived clinical surgical specimen of tumor tissue into primary single cells;

(2) staining the primary cells obtained by dissociation; and

(3) injecting the primary single cells obtained in the step (2) into the yolk sac of zebrafish embryos.

The dissociation described in the step (1) in the construction method includes: aseptically cleaning the sample in physiological saline, then cutting into small pieces in a phosphate buffer solution, trypsinizing to complete dissociation, and centrifuging to remove trypsin. For the staining in the step (2), the dye used is CM-Dil, the dye concentration is 1-5 μg/ml, the staining time is 1-10 hours, the dye is removed after staining, and the cells are washed with a phosphate buffer solution and re-suspended to a cell density of 5×10³-5×10⁵ cell/μl. The injection in the step (3) includes: immobilizing the zebrafish embryos 36-60 hours after fertilization, and injecting 10-30 nl and preferably 20 nl of the primary cells obtained in the step (2) into the yolk sac of the zebrafish embryos using a microinjector under a stereoscope.

The construction method according to the present invention further includes, after the step (3), an observation step in which qualitative analysis or/and quantitative analysis is performed under a fluorescence microscope. The metastasis and spread of the fluorescent cells in the zebrafish are observed under a fluorescence microscope after the zebrafish embryos are anesthetized with tricaine within 1-7 days after the cells are xenografted.

In the construction method of the present invention, more particularly, the step (1) includes specifically: washing the clinical surgical specimen of gastric cancer tissue twice with a phosphate buffer solution, and cutting it into small pieces of 1 mm³ by using a surgical scissor, digesting for 10-120 min with 0.25% trypsin at 37° C., centrifuging after the tissue mass is observed to be completely dissociated, and then removing trypsin.

The final concentration of the CM-Dil dye in the step (2) is 2 μg/ml, and the staining time is 1-10 hours. The dye is removed by centrifugation, and the cells are washed with a phosphate buffer and re-suspended to a cell density of 5×10³-5×10⁵ cells/0.

The step (3) includes specifically: fixing the zebrafish embryos 36-60 hours after fertilization, and injecting 10-30 nl and preferably 20 nl of the primary cells obtained in the step (2) into the yolk sac of zebrafish embryos using a microinjector under a stereoscope. The zebrafish used in the present invention is an internationally accepted model vertebrate having genes that are highly homologous (>85%) to the human genes, is a classical developmental biological research model, and can also be used as a common animal model for drug activity screening, drug toxicity evaluation, and human disease research.

The use of the tumor cell xenograft model in zebrafish of the present invention in screening drugs for gastric cancer can accurately screen out the patients that 5-FU (5-fluorouracil) is effective and patients that 5-FU (5-fluorouracil) is ineffective, providing accurate guidance for clinical medication. The use of the tumor cell xenograft model in zebrafish of the present invention in screening drugs for lung cancer can accurately screen out the patients that gefitinib, cisplatin or docetaxel is effective and patients that gefitinib, cisplatin or docetaxel is ineffective, providing accurate guidance for clinical medication.

The CM-Dil of the present invention is a dye which labels the cells by binding to a lipid molecule on the membrane structure, and has strong and stable red fluorescence (excitation peak 553 nm/emission peak 570 nm). Unlike Dil, CM-Dil has good water solubility, so it is more convenient and efficient for cell staining. The CM group (i.e., chloromethyl substituent) can react with the sulfhydryl group on the polypeptide and protein to keep the molecule stable in the aldehydes, so the CM-Dil-labeled cells can be fixed, cracked and paraffin-embedded without affecting the fluorescence. Therefore, it is an ideal fluorescent cell labeling dye for immunofluorescence, immunohistochemistry and in situ hybridization. In addition, CM-Dil is non-toxic to cells, is stable and long-lasting, and can well trace cells for a long time. Studies have confirmed that after labeling with CM-Dil, the fluorescence is stably expressed in the cells, and the positive labeling rate is over 98%. The labeled cells have good morphology, and the differentiation of cells in vitro can be effectively observed; or the labeled cells can be injected into the body, to effectively show the migration and differentiation of transplanted cells in living tissues. CM-Dil has a chemical name below: 3H-Indolium,5-[[[4-(chloromethyl)benzoyl]amino]methyl]-2-[3-(1,3-dihydro-3,3-dimethyl-1-octadecyl-2H-indol-2-ylidene)-1-propenyl]-3,3-dimethyl-1-octadecyl-, chloride.

The zebrafish used in the present invention has the characteristics of small volume, fast growth, and transparent body during the early development. The zebrafish-based PDX model has the advantages of low cost, high throughput, simple operation, and easy observation in vivo. More importantly, the experimental period with the zebrafish-based PDX model is short and is only one week. Therefore, the zebrafish-based PDX model is currently the only animal model that is expected to satisfy the clinical demand to guide individualized medications for solid tumors such as gastric cancer and lung cancer in real time.

The present invention can be used to screen for therapeutic drugs effective for tumors, particularly to remove drugs that have no therapeutic effect on patients, by constructing a patient-derived gastric cancer xenograft model in zebrafish. The patient-derived tumor xenograft (PDX) model of the present invention has higher accuracy in guiding the personalized medication of patients with gastric cancer in clinic than the human tumor (stomach cancer, lung cancer, etc.) cell line xenograft model.

In the method of the present invention, a fluorescence method is used to effectively evaluate a tumor treatment drug, and the method is simple and effective, and can meet clinical needs. The zebrafish model provided in the present invention provides a simple and effective method for evaluating the therapeutic effects of 5-FU (5-fluorouracil) on patients with gastric cancer or gefitinib, cisplatin or docetaxel on patients with lung cancer alone or in combination.

### BRIEF DESCRIPTION OF THE DRAWINGS

FIG. 1 shows the phenotype of zebrafish embryos after patient-derived primary gastric cancer cells are injected in Example 1.

FIG. 2 is a diagram showing the anticancer effects of 5-FU evaluated in two 5-FU non-sensitive patients #1 and #2-derived gastric cancer cell xenograft models in zebrafish in Example 2 of the present invention.

FIG. 3 is a diagram showing the anticancer effects of 5-FU evaluated in two 5-FU sensitive patients #3 and #4-derived gastric cancer cell xenograft models in zebrafish in Example 2 of the present invention.

FIG. 4 is a diagram showing the anticancer effects of 5-FU evaluated in two human gastric cancer cell line xenograft models in zebrafish.

FIG. 5 shows the phenotype of a patient-derived gastric cancer cell xenograft model in zebrafish treated with curcumin in Example 4 of the present invention.

FIG. 6 is a diagram showing the anticancer effect of curcumin evaluated in a patient-derived gastric cancer cell xenograft model in zebrafish in Example 4 of the present invention.

FIG. 7 shows the phenotype of zebrafish embryos after patient-derived primary lung cancer cells are injected in Example 5.

FIG. 8 is a diagram showing the anticancer effect of cisplatin in combination with docetaxel evaluated in patients *1 and *2-derived lung cancer cell xenograft models in zebrafish in Example 6 of the present invention.

FIG. 9 is a diagram showing the anticancer effect of cisplatin in combination with docetaxel evaluated in patients *3 and *4-derived lung cancer cell xenograft models in zebrafish in Example 6 of the present invention.

FIG. 10 is a diagram showing the anticancer effect of gefitinib evaluated in patients *5 and *6-derived lung cancer cell xenograft models in zebrafish in Example 7 of the present invention.

FIG. 11 is a diagram showing the anticancer effect of gefitinib evaluated in patients *7 and *8-derived lung cancer cell xenograft models in zebrafish in Example 7 of the present invention.

FIG. 12 is a diagram showing the anticancer effects of gefitinib evaluated in two human lung cancer cell line xenograft models in zebrafish in Example 8.

### DETAILED DESCRIPTION

The present invention is described in detail below with reference to the embodiments and the accompanying drawings, but the following examples should not be construed as limiting the scope of the present invention.

The methods given in examples below are all conventional methods, unless it is otherwise stated. The experimental method can also reflect the difference in accuracy between the patient-derived gastric or long cancer xenograft (PDX) model and the human gastric or lung cancer cell line xenograft model in guiding the personalized medication of patients with gastric or lung cancer in clinic.

### Example 1: Construction of a Patient-Derived Gastric Cancer Cell Xenograft Model in Zebrafish of the Present Invention

1. Isolation of Primary Cells from Gastric Cancer Tissue

The patient-derived clinical tissue biopsy that was a surgical specimen of gastric cancer was placed in physiological saline. The blood clot, necrotic tissue, fat and connective tissues on the surface of the tumor tissue were removed under aseptic conditions. The tissue was cut by an ophthalmic scissor after sterilization, and washed 2 times with sterile phosphate buffer (pH 7.4). A small amount of phosphate buffer was added, and the tissue was repeatedly cut with an ophthalmic scissor until the tissue became a paste and was about 1 mm³. 0.25% trypsin was added and the tissue was digested at 37° C. for 10 minutes. After the tissue mass was observed to be completely dissociated, centrifugation was performed to remove trypsin. The cells were re-suspended in RPMI-1640 medium containing 10% FBS (fetal calf serum).

2. Staining of Primary Cells

The primary cells obtained by dissociation were stained with CM-Dil, where the final dye concentration was 2 μg/ml, and the staining time was 1 hour. The dye was removed by centrifugation, and the cells were washed with a phosphate buffer and re-suspended to a cell density of 1×10⁴/μl.

3. Cell Transplantation

The stained cells were filled into a microinjector. The zebrafish embryos were immobilized 48 hours postfertilization, and 20 nl of the primary cells obtained in the step (2) into the yolk sac of zebrafish embryos using the microinjector under a stereoscope.

4. Observation Under Fluorescence Microscope

Within 7 days after the injection, the growth, metastasis and spread of the cells derived from the patient in the zebrafish were observed under a fluorescence microscopy and photographed.

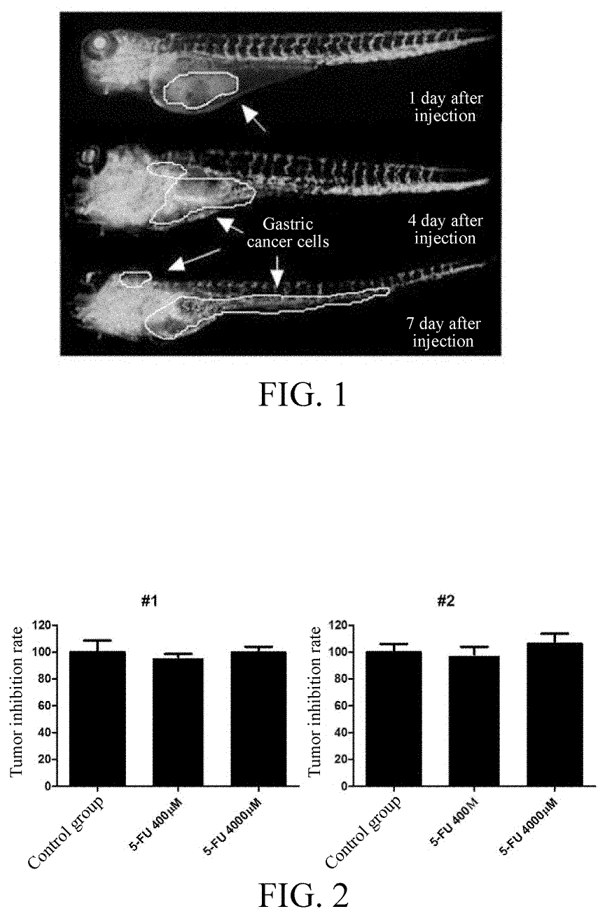

As shown in FIG. 1, the patient-derived gastric cancer cells display a proliferative and spreading phenotype in zebrafish embryos. 4 days after the injection, it can be seen that the patient-derived gastric cancer cells had spread to the abdomen and the head. 7 days after the injection, it can be seen that the patient-derived gastric cancer cells had spread to the tail and the brain of the zebrafish embryos.

### Example 2: Evaluation of the Clinical Anticancer Effect of 5-FU with 4 Patient-Derived Xenograft Zebrafish Models

1. Determination of Safe Dose

Two days after fertilization, the zebrafish embryos were treated (soaked) with various concentrations of 5-FU for three days, and the highest 5-FU concentration within the safety range for embryos was determined to be 4000 nM.

2. Drug Treatment of Zebrafish Embryos

The 4000-μM and 400 μM 5-FU were used to soak the zebrafish embryo model injected with the primary gastric cancer cells derived from different patients prepared by the method of Example 1 for three days, and 0.1% DMSO was used as a solvent control.

3. Observation of Antitumor Effect Under Fluorescence Microscope

The proliferation and spread of red patient-derived cells in the zebrafish embryos were observed after treatment. The red cells were photographed under a fluorescence microscope, and the red fluorescence intensity was quantified by Image Pro Plus software to calculate the anti-tumor effect of 5-FU by a formula: Tumor inhibition rate of the drug=(the fluorescence intensity of the treatment group/the fluorescence intensity of the control group)*100% (see FIGS. 2 and 3).

The results with the patient-derived gastric cancer xenograft model in zebrafish show that at day 7 after the injection, the gastric cancer cells derived from #1 and #2 patients with gastric cancer are not sensitive to 5-FU, and there is no obvious tumor inhibition effect; and the clinical effect of 5-FU in the treatment of the two patients with gastric cancer is not significant either. The gastric cancer cells derived from #3 and #4 patients with gastric cancer are sensitive to 5-FU, and the tumor proliferation is significantly inhibited; and the clinical symptoms of these two gastric cancer patients were significantly improved after treatment with 5-FU.

Therefore, the drug evaluation results with the patient-derived gastric cancer xenograft models in zebrafish are highly correlated with the clinical outcomes.

### Example 3: Evaluation of the Anticancer Effects of 5-FU with Two Human Gastric Cancer Cell Line (SGC-7901 and AGS) Xenograft Models in Zebrafish

1. Drug Treatment of Zebrafish Embryos

4000 μM and 400 μM5-FU were used to soak the zebrafish embryos injected with gastric cancer cell lines (SGC-7901 and AGS) (constructed as in Example 1) for three consecutive days, and 0.1% DMSO was used as a solvent control.

2. Observation of Antitumor Effect Under Fluorescence Microscope

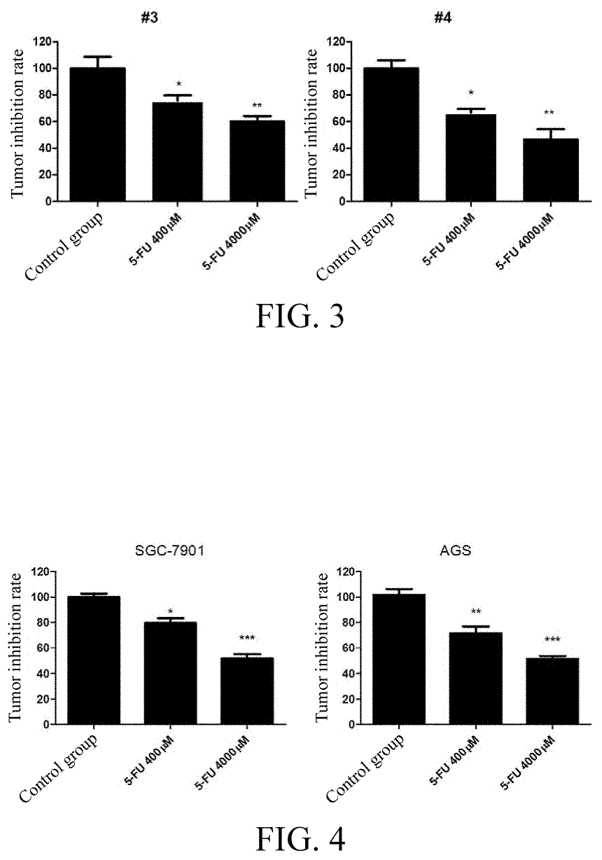

The proliferation and spread of red patient-derived cells in the zebrafish embryos were observed after treatment. The red cells were photographed under a fluorescence microscope, and the red fluorescence intensity was quantified by Image Pro Plus software to calculate the anti-tumor effect of 5-FU by a formula: Tumor inhibition rate of the drug=(the fluorescence intensity of the treatment group/the fluorescence intensity of the control group)*100% (see FIG. 4).

The results with human gastric cancer cell line xenograft models in zebrafish show that both gastric cancer cell lines are sensitive to 5-FU at day 7 after injection. Therefore, the clinical anti-gastric cancer effects of the drug 5-FU evaluated with the xenograft zebrafish models established with the cell lines differ from the actual clinical efficacy, and cannot be used to guide the clinical medication of gastric cancer.

### Example 4: Evaluation of the Anti-Gastric Cancer Effect of Curcumin with Patient-Derived Gastric Cancer Xenograft Animal Models

1. Four groups, each group having four randomly selected zebrafish embryos that were two days old after fertilization, were placed in a solution of curcumin containing 0.1% DMSO. The curcumin concentrations were 10 μM, 30 μM, 50 μM, and 70 μM, respectively, and the treatment was continued for 3 days. The death of the zebrafish embryos was observed, and the highest concentration of curcumin in the safe range for the embryos was determined to be 50 μM.

2. The zebrafish embryos that have been injected with the patient-derived primary gastric cancer cells were placed in 10 μM and 50 μM aqueous solutions of curcumin containing 0.1% DMSO as described in the step 1, for three consecutive days. The zebrafish embryos that have been injected with humanized gastric cancer primary cells were placed in an aqueous 0.1% DMSO solution for three consecutive days and taken as a solvent control group.

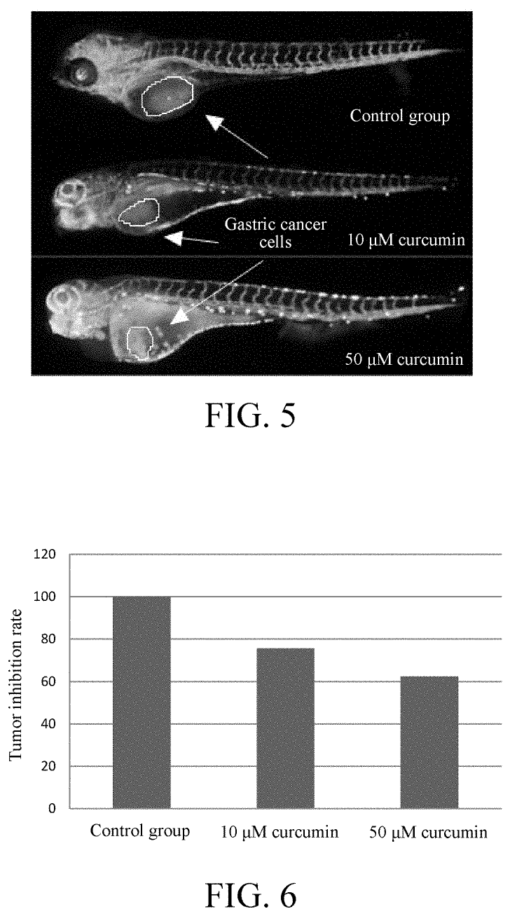

3. The proliferation and spread of patient-derived gastric cancer cells in the zebrafish embryos were observed after treatment. In this example, the gastric cells are proliferated in situ. The human-derived gastric cells are photographed under a fluorescence microscope (FIG. 5), the fluorescence intensity is quantified using Image Pro Plus software, and the antitumor effect of curcumin is calculated (FIG. 6).

### Example 5: Construction of a Patient-Derived Lung Cancer Cell Xenograft Model in Zebrafish of the Present Invention

1. Isolation of Primary Cells from Lung Cancer Tissue

The patient-derived clinical tissue biopsy that was a surgical specimen of lung cancer was placed in physiological saline. The blood clot, necrotic tissue, fat and connective tissues on the surface of the tumor tissue were removed under aseptic conditions. The tissue was cut by an ophthalmic scissor after sterilization, and washed 2 times with sterile phosphate buffer (pH 7.4). A small amount of phosphate buffer was added, and the tissue was repeatedly cut with an ophthalmic scissor until the tissue became a paste and was about 1 mm³ in size. 0.25% trypsin was added and the tissue was digested at 37° C. for 10 minutes. After the tissue mass was observed to be completely dissociated, centrifugation was performed to remove trypsin. The cells were resuspended in RPMI-1640 medium containing 10% FBS (fetal calf serum).

2. Staining of Primary Cells

The primary cells obtained by dissociation were stained with CM-Dil, where the final dye concentration was 2 μg/ml, and the staining time was 1 hour. The dye was removed by centrifugation, and the cells were washed with a phosphate buffer and re-suspended to a cell density of 1×10⁴/μl.

3. Cell Transplantation

The stained cells were filled into a microinjector. The zebrafish embryos were immobilized 48 hours after fertilization, and 20 nl of the primary cells obtained in the step (2) into the yolk sac of zebrafish embryos using the microinjector under a stereoscope.

4. Observation Under Fluorescence Microscope

Within 4 days after the injection, the growth, metastasis and spread of the cells derived from the patient in the zebrafish were observed under a fluorescence microscopy and photographed.

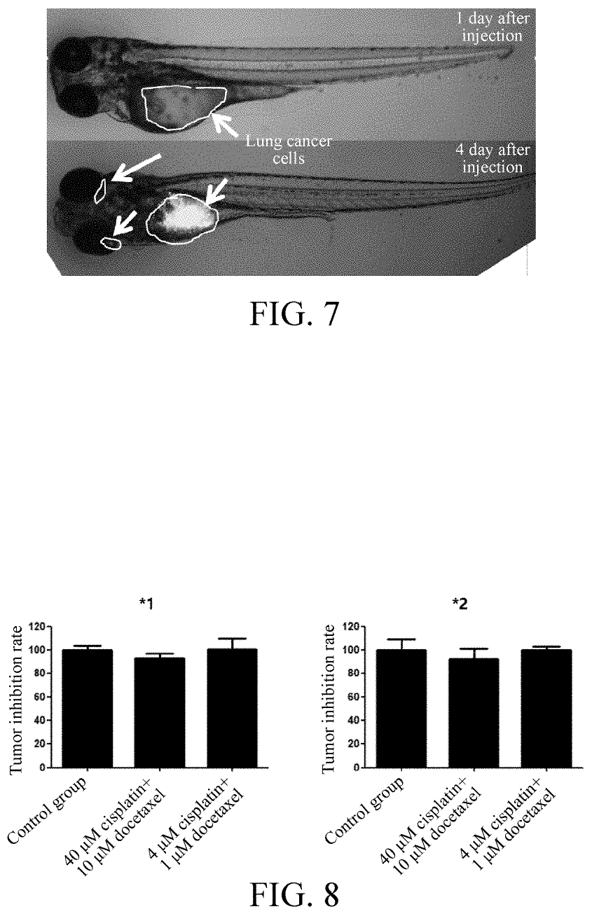

As shown in FIG. 7, the patient-derived lung cancer cells display a proliferative and spreading phenotype in zebrafish embryos. 4 days after the injection, it can be seen that the patient-derived lung cancer cells had spread to the abdomen and the head.

### Example 6: Evaluation of the Clinical Anticancer Effect of Docetaxel+Cisplatin with 4 Patient-Derived Lung Cancer Xenograft Models in Zebrafish. Determination of Safe Dose

Two days after fertilization, the zebrafish embryos were treated (soaked) respectively with various concentrations of cisplatin and docetaxel for three days, and the highest cisplatin concentration is 40 μM within the safety range for embryos was determined to be 40 μM and the docetaxel concentration was 10 μM.

2. Drug Treatment of Zebrafish Embryos

The 40-μM cisplatin+10-μM docetaxel and 4-μM cisplatin+1-μM docetaxel were used to soak the zebrafish embryo model injected with the primary lung cancer cells derived from different patients prepared by the method of Example 1 for three consecutive days, and 0.1% DMSO was used as a solvent control. The zebrafish embryo models with the same patient-derived lung cancer primary cells were treated with two different concentrations of the agents.

3. Observation of Antitumor Effect Under Fluorescence Microscope

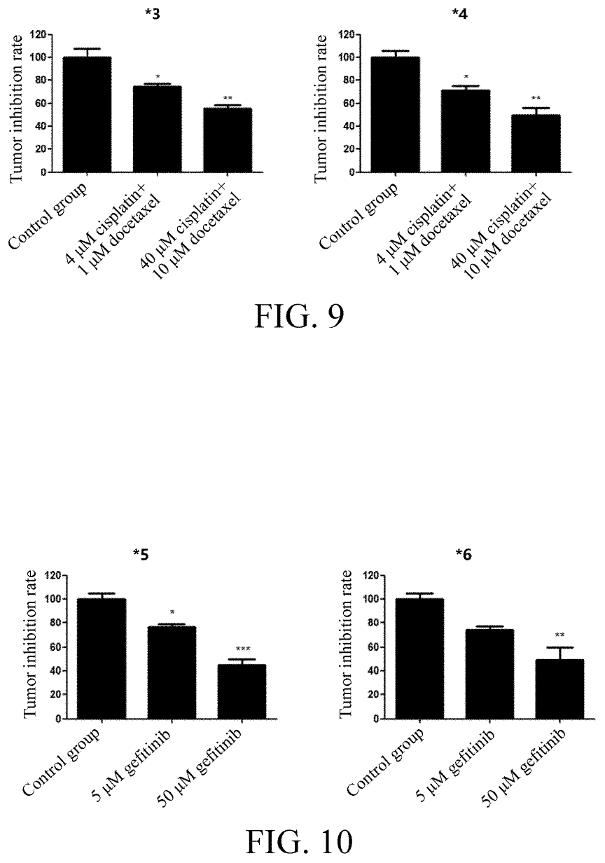

The proliferation and spread of red patient-derived cells in the zebrafish embryos were observed after treatment. The red cells were photographed under a fluorescence microscope, and the red fluorescence intensity was quantified by Image Pro Plus software to calculate the anti-tumor effect of the drug by a formula: Tumor inhibition rate of the drug=(the fluorescence intensity of the treatment group/the fluorescence intensity of the control group)*100% (see FIGS. 8 and 9).

The results with the patient-derived lung cancer xenograft model in zebrafish show that at day 7 after the injection, the lung cancer cells derived from *1 and *2 patients with lung cancer are not sensitive to the combination of cisplatin+docetaxel, and there is no obvious tumor inhibition effect; and the clinical effect of cisplatin+docetaxel in the treatment of the two patients with lung cancer is not significant either. The lung cancer cells derived from *3 and *4 patients with lung cancer are sensitive to the combination of cisplatin+docetaxel, and the tumor proliferation is significantly inhibited; and the clinical symptoms of these two lung cancer patients were significantly improved after treatment with the two drugs in combination.

Therefore, the drug evaluation results with the patient-derived lung cancer xenograft models in zebrafish are highly correlated with the clinical outcomes. The experimental results of multiple groups also confirm this conclusion.

### Example 7: Evaluation of the Clinical Anticancer Effect of Gefitinib with 4 Patient-Derived Lung Cancer Cell Xenograft Models in Zebrafish

1. Determination of Safe Dose

Two days after fertilization, the zebrafish embryos were treated (soaked) with various concentrations of gefitinib for three consecutive days, and the highest gefitinib concentration within the safety range for embryos was determined to be 50 μM.

2. Drug Treatment of Zebrafish Embryos

The 50-μM and 5-μM gefitinib were used to soak the zebrafish embryo model injected with the patient-derived primary lung cancer cells prepared by the method of Example 1 for three consecutive days, and 0.1% DMSO was used as a solvent control. The zebrafish embryo models with the same patient-derived lung cancer primary cells were treated with two different concentrations of the agents.

3. Observation of Antitumor Effect Under Fluorescence Microscope

The proliferation and spread of red patient-derived cells in the zebrafish embryos were observed after treatment. The red cells were photographed under a fluorescence microscope, and the red fluorescence intensity was quantified by Image Pro Plus software to calculate the anti-tumor effect of gefitinib by a formula: Tumor inhibition rate of the drug=(the fluorescence intensity of the treatment group/the fluorescence intensity of the control group)*100% (see FIGS. 10 and 11).

The results with the patient-derived lung cancer xenograft model in zebrafish show that at day 7 after the injection, the lung cancer cells derived from *5 and *6 patients with lung cancer are sensitive to gefitinib, there is obvious tumor inhibition effect, and the tumor proliferation is obviously inhibited; and the clinical effect of gefitinib in the treatment of the two patients with lung cancer is also significant. The lung cancer cells derived from *7 and *8 patients with lung cancer are not sensitive to gefitinib; and the clinical symptoms of these two lung cancer patients are not significantly improved after treatment with the two drugs in combination.

Therefore, the drug evaluation results with the patient-derived lung cancer xenograft models in zebrafish are highly correlated with the clinical outcomes. The experimental results of multiple groups also confirm this conclusion.

### Example 8: Evaluation of the Anticancer Effects of Gefitinib with Two Human Lung Cancer Cell Line (A549 and HCC827) Xenograft Models in Zebrafish

1. Drug Treatment of Zebrafish Embryos 50 μM gefitinib and 5 μM gefitinib were used to soak zebrafish embryos (constructed in accordance with the method of Example 1) that have been injected with lung cancer cell lines (A549 and HCC827) for three consecutive days and 0.1% DMSO was used as a solvent control.

2. Observation of Antitumor Effect Under Fluorescence Microscope

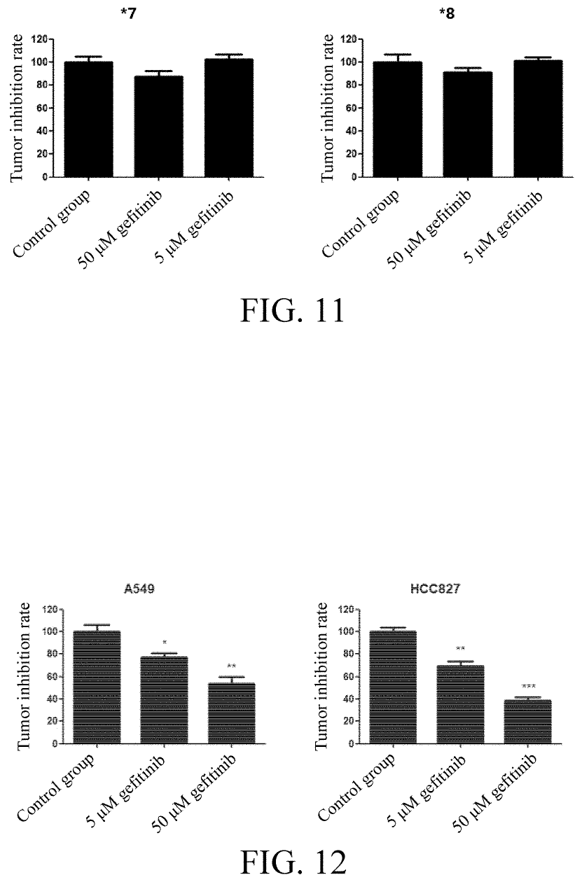

The proliferation and spread of red patient-derived cells in the zebrafish embryos were observed after treatment. The red cells were photographed under a fluorescence microscope, and the red fluorescence intensity was quantified by Image Pro Plus software to calculate the anti-tumor effect of gefitinib by a formula: Tumor inhibition rate of the drug=(the fluorescence intensity of the treatment group/the fluorescence intensity of the control group)*100% (see FIG. 12).

The results with human lung cancer cell line xenograft models in zebrafish show that both lung cancer cell lines are sensitive to gefitinib at day 7 after injection. Therefore, the clinical anti-lung cancer effects of the drug gefitinib evaluated with the xenograft zebrafish models established with the cell lines differ from the actual clinical efficacy, and cannot be used to guide the clinical medication of lung cancer.

1. A patient-derived tumor cell xenograft model comprising a zebrafish embryo transplanted with primary single cells isolated and cultured from tumor tissue derived from a patient.

2. The model according to claim 1, wherein the transplantation is carried out within 24-72 hours after fertilization.

3. The model according to claim 1, wherein the transplantation site is in the yolk sac of the embryos.

4. The model according to claim 1, wherein the primary single cells are stained.

5. The model according to claim 1, wherein the tumor is selected from gastric cancer and lung cancer.

6. (canceled)

7. A method for studying the mechanism underlying the proliferation, metastasis, spread or drug resistance of tumors, or in screening effective therapeutic drugs for tumors using the model according to claim 1, the method comprising the steps of:
determining the highest drug concentration within the safe range of a tumor drug candidate for the embryos;
treating the zebrafish embryos with the drug candidate having a drug concentration in the safe range, and taking the solvent for dissolving the drug candidate as a control and undergoing the same process; and
performing qualitative analysis or/and quantitative analysis of the proliferation and spread of the patient-derived cells in the zebrafish embryos under a fluorescence microscope.

8. The method according to claim 7, wherein for the quantitative analysis, the antitumor effect is calculated according to the following formula:
the tumor inhibition rate of the drug=(the fluorescence intensity of the treatment group/the fluorescence intensity of the control group)*100%.

9. The method according to claim 7, wherein the treatment with the drug candidate is continued for 2 to 5 days.

10. The method according to claim 7, wherein the tumor is selected from gastric cancer and the drug is selected from 5-fluorouracil.

11. The method according to claim 7, wherein the tumor is selected from lung cancer and the drug is one selected from gefitinib, cis-cisplatin or docetaxel, or a combination of two thereof.

12. A method for constructing a patient-derived tumor cell xenograft model in zebrafish, comprising the steps of:
(1) dissociating a patient-derived clinical surgical specimen of tumor tissue into primary single cells;
(2) staining the primary cells obtained by dissociation; and
(3) injecting the primary single cells obtained in the step (2) into the yolk sac of zebrafish embryos.

13. The construction method according to claim 12, wherein
the dissociation in the step (1) comprises aseptically cleaning the sample in physiological saline, then cutting into small pieces in a phosphate buffer solution, trypsinizing to complete dissociation, and centrifuging to remove trypsin;
for the staining in the step (2), the dye used is CM-Dil, the dye concentration is 1-5 μg/ml, the staining time is 1-10 hours, the dye is removed after staining, and the cells are washed with a phosphate buffer solution and resuspended to a cell density of 5×10³-5×10⁵ cell/μ; and
the injection in the step (3) comprises: immobilizing the zebrafish embryos 36-60 hours after fertilization, and injecting 10-30 nl of the primary single cells obtained in the step (2) into the yolk sac of the zebrafish embryos using a microinjector under a stereoscope.

14. The construction method according to claim 12, further comprising, after the step (3), a step of performing qualitative analysis or/and quantitative analysis under a fluorescence microscope.

15. The construction method according to claim 12, wherein the metastasis and spread of the fluorescent cells in the zebrafish are observed under a fluorescence microscope after the zebrafish embryos are anesthetized with tricaine within 1-7 days after the cells are xenografted.

16. The model according to claim 2, wherein the transplantation is carried out within 36-60 hours after fertilization.

17. The model according to claim 2, wherein the transplantation is carried out within 48 hours after fertilization.

18. The model according to claim 4, wherein the primary cells are stained with CM-Dil.

19. The use according to claim 9, wherein the treatment with the drug candidate is continued for at least 3 days.

20. The construction method according to claim 13, wherein
the injection in the step (3) comprises: immobilizing the zebrafish embryos 36-60 hours after fertilization, and injecting at least 20 nl of the primary single cells obtained in the step (2) into the yolk sac of the zebrafish embryos using a microinjector under a stereoscope.
